(19) United States
(12) Patent Application Publication
(10) Pub. No.: US 2019/0374111 A1
(43) Pub. Date: Dec. 12, 2019

(54) TICK DETECTION USING THERMAL IMAGING

(71) Applicant: James Francis White, Medfield, MA (US)

(72) Inventor: James Francis White, Medfield, MA (US)

(21) Appl. No.: 16/438,586

(22) Filed: Jun. 12, 2019

Related U.S. Application Data
(60) Provisional application No. 62/683,820, filed on Jun. 12, 2018.

(51) Int. Cl. A61B 5/01; A01N 47/00; A01K 13/00

(52) U.S. Cl. CPC A61B 5/01; A01K 13/00; A01N 47/00

(57) ABSTRACT

A computer-implemented method, thermographic system, and computer program product may include receiving, on a computing device, a thermal image of a host body. The thermal image may be captured by a thermographic camera. A presence of a cold-blooded animal on the host body may be detected based upon temperature information from the thermal image.

### CROSS-REFERENCE TO RELATED APPLICATION

This application claims the benefit of U.S. provisional patent application Ser. No. 62/683,820, entitled “Tick Detection Using Thermal Imaging,” filed on Jun. 12, 2018, the entire disclosure of which is incorporated herein by reference.

### FIELD OF THE INVENTION

The present invention generally relates to a method and system for utilizing thermal imaging techniques to identify heat signatures of mammals compared to heat signatures associated with cold blooded animals.

### BACKGROUND OF THE INVENTION

Ticks are ectoparasites that thrive in heavily wooded or grassy areas, but easily adapt to any number of different environments around the world. Generally, ticks live close to areas populated by land animals, including wildlife, domesticated animals and unfortunately, humans. While the specific behavior patterns and “hunting” strategies differ based on the type of tick, once attached to a host, a tick will eventually attempt to work its way to the surface of the skin and cement its head beneath the skin's surface into direct fluid communication with the host's bloodstream. If not quickly removed, the tick will feed on the host's blood, during which time the tick may transmit disease carrying organisms to the host. As tick-borne diseases increasingly threaten the health of unsuspecting victims, tools for detecting ticks on a host body are imperative to preventing life threatening illnesses.

### BRIEF SUMMARY OF DISCLOSURE

In one example implementation, a computer-implemented method may include receiving a thermal image of a host body. The computer-implemented method may also include detecting the presence of a cold-blooded animal on the host body based upon temperature information from the thermal image.

One or more of the following example features may be included. Detecting the presence of the cold-blooded animal on the host body may include identifying a localized region of temperature differential in the thermal image of the host body. Detecting the presence of the cold-blooded animal on the host body may include identifying a location of the cold-blooded animal on the host body based on the localized region of temperature differential. Identifying the location of the cold-blooded animal on the host body may include utilizing a temperature delta between the host body and the cold-blooded animal. In response to detecting the presence of the cold-blooded animal on the host body, the computer-implemented method may include providing a notification to a user. Detecting the presence of the cold-blooded animal on the host body may include detecting the localized region of temperature differential having a size corresponding to a size of the cold-blooded animal of interest. Identifying the location of the cold-blooded animal on the host body may include detecting one or more localized regions exhibiting a lower temperature than the host body. The one or more localized regions exhibiting a lower temperature than the host body may be selected based upon, at least in part, the thermal temperature of the host body.

In another example implementation, a thermographic system may include a thermographic camera. The thermographic camera may be configured to capture a thermal image of a host body. The thermographic system may also include a computing device for receiving the thermal image of the host body. The computing device may also detect the presence of a cold-blooded animal on the host body based upon temperature information from the thermal image.

One or more of the following example features may be included. Detecting the presence of the cold-blooded animal on the host body may include identifying a localized region of temperature differential in the thermal image of the host body. Detecting the presence of the cold-blooded animal on the host body may include identifying a location of the cold-blooded animal on the host body based on the localized region of temperature differential. Identifying the location of the cold-blooded animal on the host body may include utilizing a temperature delta between the host body and the cold-blooded animal. In response to detecting the presence of the cold-blooded animal on the host body, the computer-implemented method may include providing a notification to a user. Detecting the presence of the cold-blooded animal on the host body may include detecting the localized region of temperature differential having a size corresponding to a size of the cold-blooded animal of interest. Identifying the location of the cold-blooded animal on the host body may include detecting one or more localized regions exhibiting a lower temperature than the host body. The one or more localized regions exhibiting a lower temperature than the host body may be selected based upon, at least in part, the thermal temperature of the host body.

In another example implementation, a computer program product may reside on a computer readable storage medium having a plurality of instructions stored thereon which, when executed across one or more processors, may cause at least a portion of the one or more processors to perform operations, that may include but are not limited to, receiving a thermal image of a host body. Detecting the presence of a cold-blooded animal on the host body may be based upon temperature information from the thermal image.

One or more of the following example features may be included. Detecting the presence of the cold-blooded animal on the host body may include identifying a localized region of temperature differential in the thermal image of the host body. Detecting the presence of the cold-blooded animal on the host body may include identifying a location of the cold-blooded animal on the host body based on the localized region of temperature differential. Identifying the location of the cold-blooded animal on the host body may include detecting one or more localized regions exhibiting a lower temperature than the host body.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1 is an example diagrammatic view of a thermographic system coupled to an example distributed computing network according to one or more example implementations of the disclosure;

FIG. 2 is an example diagrammatic view of a computing device of FIG. 1 according to one or more example implementations of the disclosure;

FIG. 3 is an example flowchart of a thermographic system according to one or more example implementations of the disclosure; and

FIG. 4 is an illustrative example of a thermal image depicting the presence of a cold-blooded animal on the host body based upon temperature information from the thermal image.

Like reference symbols in the various drawings indicate like elements.

### DETAILED DESCRIPTION

Ticks are ectoparasites that thrive in heavily wooded or grassy areas, but easily adapt to any number of different environments around the world. Generally, ticks live close to areas populated by land animals, including wildlife, domesticated animals and unfortunately, humans. While the specific behavior patterns and “hunting” strategies differ based on the type of tick, once attached to a host, a tick will typically attempt to work its way to the surface of the skin and cement its head beneath the skin's surface into direct fluid communication with the host's bloodstream. If not quickly removed, the tick will feed on the host's blood, during which time the tick may transmit disease carrying organisms to the host. As tick-borne diseases increasingly threaten the health of unsuspecting victims, tools for detecting ticks on a host body are imperative to preventing life threatening illnesses.

Thermal Infrared Imaging, also referred to as “non-visible” imaging, functions within an infrared spectrum that is not visible to the human eye. Readily appreciated by a person of ordinary skill in the art, thermographic cameras may detect radiation in the long-infrared range of the electromagnetic spectrum (i.e., roughly 9,000-14,000 nanometers or 9-14 μm) and produce images relative to that level of radiation. Since infrared radiation is emitted by all objects with a temperature above absolute zero, thermographic cameras make it possible to see the surrounding environment with or without light. Moreover, the amount of radiation emitted by any given object increases with temperature; therefore, thermographic cameras may allow one to see variations in temperature. When viewed through a thermal imaging camera, warm objects (i.e., generally depicted within a yellow, orange and red color gradient) stand out well against cooler backgrounds (i.e., generally depicted within a green, blue, and violet color gradient). As such, humans and other warm-blooded animals become easily visible against the environment, day or night. Various examples of thermographic cameras are commercially available, e.g., as standalone devices and/or attachments for other electronic devices, e.g., smartphones or the like.

In some implementations, the present disclosure may be embodied as a method, system, or computer program product. Accordingly, in some implementations, the present disclosure may take the form of an entirely hardware implementation, an entirely software implementation (including firmware, resident software, micro-code, etc.) or an implementation combining software and hardware aspects that may all generally be referred to herein as a “circuit,” “module” or “system.” Furthermore, in some implementations, the present disclosure may take the form of a computer program product on a computer-usable storage medium having computer-usable program code embodied in the medium.

In some implementations, any suitable computer usable or computer readable medium (or media) may be utilized. The computer readable medium may be a computer readable signal medium or a computer readable storage medium. The computer-usable, or computer-readable, storage medium (including a storage device associated with a computing device or client electronic device) may be, for example, but is not limited to, an electronic, magnetic, optical, electromagnetic, infrared, or semiconductor system, apparatus, device, or any suitable combination of the foregoing. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a digital versatile disk (DVD), a static random access memory (SRAM), a memory stick, a floppy disk, a mechanically encoded device such as punch-cards or raised structures in a groove having instructions recorded thereon, a media such as those supporting the internet or an intranet, or a magnetic storage device. Note that the computer-usable or computer-readable medium could even be a suitable medium upon which the program is stored, scanned, compiled, interpreted, or otherwise processed in a suitable manner, if necessary, and then stored in a computer memory. In the context of the present disclosure, a computer-usable or computer-readable, storage medium may be any tangible medium that can contain or store a program for use by or in connection with the instruction execution system, apparatus, or device.

In some implementations, a computer readable signal medium may include a propagated data signal with computer readable program code embodied therein, for example, in baseband or as part of a carrier wave. In some implementations, such a propagated signal may take any of a variety of forms, including, but not limited to, electro-magnetic, optical, or any suitable combination thereof. In some implementations, the computer readable program code may be transmitted using any appropriate medium, including but not limited to the internet, wireline, optical fiber cable, RF, etc. In some implementations, a computer readable signal medium may be any computer readable medium that is not a computer readable storage medium and that can communicate, propagate, or transport a program for use by or in connection with an instruction execution system, apparatus, or device.

In some implementations, computer program code for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java®, Smalltalk, C++ or the like. Java® and all Java-based trademarks and logos are trademarks or registered trademarks of Oracle and/or its affiliates. However, the computer program code for carrying out operations of the present disclosure may also be written in conventional procedural programming languages, such as the “C” programming language, PASCAL, or similar programming languages, as well as in scripting languages such as Javascript, PERL, or Python. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the internet using an Internet Service Provider). In some implementations, electronic circuitry including, for example, programmable logic circuitry, field-programmable gate arrays (FPGAs) or other hardware accelerators, micro-controller units (MCUs), or programmable logic arrays (PLAs) may execute the computer readable program instructions/code by utilizing state information of the computer readable program instructions to personalize the electronic circuitry, in order to perform aspects of the present disclosure.

In some implementations, the flowchart and block diagrams in the figures illustrate the architecture, functionality, and operation of possible implementations of apparatus (systems), methods and computer program products according to various implementations of the present disclosure. Each block in the flowchart and/or block diagrams, and combinations of blocks in the flowchart and/or block diagrams, may represent a module, segment, or portion of code, which comprises one or more executable computer program instructions for implementing the specified logical function(s)/act(s). These computer program instructions may be provided to a processor of a general purpose computer, special purpose computer, or other programmable data processing apparatus to produce a machine, such that the computer program instructions, which may execute via the processor of the computer or other programmable data processing apparatus, create the ability to implement one or more of the functions/acts specified in the flowchart and/or block diagram block or blocks or combinations thereof. It should be noted that, in some implementations, the functions noted in the block(s) may occur out of the order noted in the figures (or combined or omitted). For example, two blocks shown in succession may, in fact, be executed substantially concurrently, or the blocks may sometimes be executed in the reverse order, depending upon the functionality involved.

In some implementations, these computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks or combinations thereof.

In some implementations, the computer program instructions may also be loaded onto a computer or other programmable data processing apparatus to cause a series of operational steps to be performed (not necessarily in a particular order) on the computer or other programmable apparatus to produce a computer implemented process such that the instructions which execute on the computer or other programmable apparatus provide steps for implementing the functions/acts (not necessarily in a particular order) specified in the flowchart and/or block diagram block or blocks or combinations thereof.

As generally discussed above, in some implementations a thermographic system consistent with the present disclosure may be implemented, at least in part, by one or more computing devices. For example, the entire thermographic system may be implemented by a single computing device, and/or by more than one computing devices, each implementing all or a portion of the thermographic system. In an implementation in which the thermographic system is implemented by more than one computing device, the various computing devices may be in communication with one another, e.g., via any suitable wired or wireless communication protocol, and/or by way of a shared medium, such as a shared storage medium, which may be simultaneously and/or sequentially in communication with each of the various computing device. For example, and as explained in greater detail below, a first electronic device (e.g., which may include a thermographic imaging device, including, but not limited to, a standalone thermographic camera and/or a computing device having thermographic imaging capabilities) may capture a thermal image of a host body. The thermal image of the host body may be processed by the first electronic device to detect the presence of a cold-blooded animal, and/or the thermal image may be transmitted to another electronic device for processed to detect the presence of a cold-blooded animal.

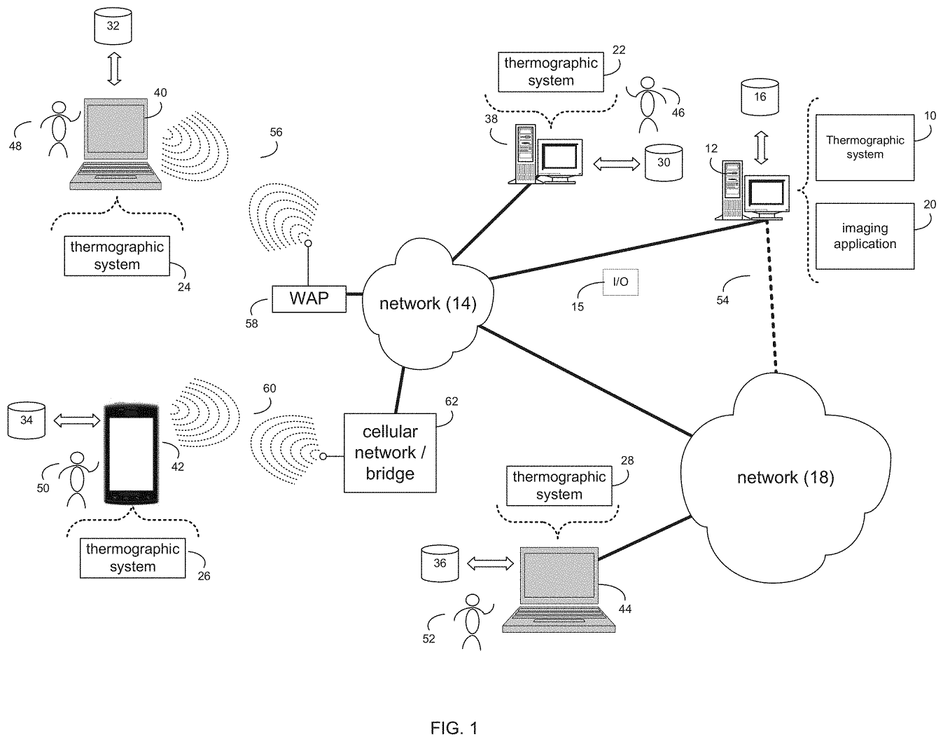

Referring now to the example implementation of FIG. 1, there is shown a thermographic system 10 that may reside on and be executed by one or more computing device, either singly and/or in combination with one or more other computing device. For example, thermographic system 10 that may reside on and may be executed by a computer (e.g., computer 12), which may, in some embodiments, be connected to a network (e.g., network 14) (e.g., the internet or a local area network). Examples of computer 12 (and/or one or more of the client electronic devices noted below) may include, but are not limited to, a storage system (e.g., a Network Attached Storage (NAS) system, a Storage Area Network (SAN)), a personal computer(s), a laptop computer(s), mobile computing device(s), smartphone(s), tablet computer(s), a server computer, a series of server computers, a mainframe computer(s), or a computing cloud(s). As is known in the art, a SAN may include one or more of the client electronic devices, including a RAID device and a NAS system. In some implementations, each of the aforementioned may be generally described as a computing device. In certain implementations, a computing device may be a physical or virtual device. In many implementations, a computing device may be any device capable of performing operations, such as a dedicated processor, a portion of a processor, a virtual processor, a portion of a virtual processor, portion of a virtual device, or a virtual device. In some implementations, a processor may be a physical processor or a virtual processor. In some implementations, a virtual processor may correspond to one or more parts of one or more physical processors. In some implementations, the instructions/logic may be distributed and executed across one or more processors, virtual or physical, to execute the instructions/logic. Computer 12 may execute an operating system, for example, but not limited to, Microsoft® Windows®; Mac® OS X®; Red Hat® Linux®, Windows® Mobile, Chrome OS, Blackberry OS, Fire OS, or a custom operating system. (Microsoft and Windows are registered trademarks of Microsoft Corporation in the United States, other countries or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

In some implementations, as will be discussed below in greater detail, a thermographic system, such as thermographic system 10 of FIG. 1, may receive a thermal image of a host body. Thermographic system 10 may also detect the presence of a cold-blooded animal on the host body and may be based upon temperature information from the thermal image.

In some implementations, the instruction sets and subroutines of thermographic system 10, which may be stored on storage device, such as storage device 16, coupled to computer 12, may be executed by one or more processors and one or more memory architectures included within computer 12. In some implementations, storage device 16 may include but is not limited to: a hard disk drive; all forms of flash memory storage devices; a tape drive; an optical drive; a RAID array (or other array); a random access memory (RAM); a read-only memory (ROM); or combination thereof. In some implementations, storage device 16 may be organized as an extent, an extent pool, a RAID extent (e.g., an example 4D+1P R5, where the RAID extent may include, e.g., five storage device extents that may be allocated from, e.g., five different storage devices), a mapped RAID (e.g., a collection of RAID extents), or combination thereof.

In some implementations, network 14 may be connected to one or more secondary networks (e.g., network 18), examples of which may include but are not limited to: a local area network; a wide area network or other telecommunications network facility; or an intranet, for example. The phrase “telecommunications network facility,” as used herein, may refer to a facility configured to transmit, and/or receive transmissions to/from one or more mobile client electronic devices (e.g., cellphones, etc.) as well as many others.

In some implementations, computer 12 may include a data store, such as a database (e.g., relational database, object-oriented database, triplestore database, etc.) and may be located within any suitable memory location, such as storage device 16 coupled to computer 12. In some implementations, data, metadata, information, etc. described throughout the present disclosure may be stored in the data store. In some implementations, computer 12 may utilize any known database management system such as, but not limited to, DB2, in order to provide multi-user access to one or more databases, such as the above noted relational database. In some implementations, the data store may also be a custom database, such as, for example, a flat file database or an XML database. In some implementations, any other form(s) of a data storage structure and/or organization may also be used. In some implementations, thermographic system 10 may be a component of the data store, a standalone application that interfaces with the above noted data store and/or an applet/application that is accessed via thermographic systems 22, 24, 26, 28. In some implementations, the above noted data store may be, in whole or in part, distributed in a cloud computing topology. In this way, computer 12 and storage device 16 may refer to multiple devices, which may also be distributed throughout the network.

In some implementations, computer 12 may execute an imaging application (e.g., imaging application 20), examples of which may include, but are not limited to imaging applications from FLIR Systems, Inc., such as the FLIR One thermal camera. It will be appreciated that various other thermographic cameras and/or other thermal imaging systems may suitably be used in connection with the present disclosure.

In some implementations, thermographic system 10 and/or imaging application 20 may be accessed via one or more of thermographic systems 22, 24, 26, 28. In some implementations, thermographic system 10 may be a standalone application, or may be an applet/application/script/extension that may interact with and/or be executed within imaging application 20, a component of imaging application 20, and/or one or more of thermographic systems 22, 24, 26, 28. In some implementations, imaging application 20 may be a standalone application, or may be an applet/application/script/extension that may interact with and/or be executed within thermographic system 10, a component of thermographic system 10, and/or one or more of thermographic systems 22, 24, 26, 28. In some implementations, one or more of thermographic systems 22, 24, 26, 28 may be a standalone application, or may be an applet/application/script/extension that may interact with and/or be executed within and/or be a component of thermographic system 10 and/or imaging application 20. Examples of thermographic systems 22, 24, 26, 28 may include, but are not limited to, e.g., imaging application 20, a standard and/or mobile web browser, an email application (e.g., an email client application), a textual and/or a graphical user interface, a customized web browser, a plugin, an Application Programming Interface (API), or a custom application. The instruction sets and subroutines of thermographic systems 22, 24, 26, 28, which may be stored on storage devices 30, 32, 34, 36, coupled to client electronic devices 38, 40, 42, 44, may be executed by one or more processors and one or more memory architectures incorporated into client electronic devices 38, 40, 42, 44.

In some implementations, one or more of storage devices 30, 32, 34, 36, may include but are not limited to: hard disk drives; flash drives, tape drives; optical drives; RAID arrays; random access memories (RAM); and read-only memories (ROM). Examples of client electronic devices 38, 40, 42, 44 (and/or computer 12) may include, but are not limited to, a drone buoy, a personal computer (e.g., client electronic device 38), a laptop computer (e.g., client electronic device 40), a smart/data-enabled, cellular phone (e.g., client electronic device 42), a notebook computer (e.g., client electronic device 44), a tablet, a server, a television, a smart television, a smart speaker, an Internet of Things (IoT) device, a media (e.g., video, photo, etc.) capturing device, and a dedicated network device. Client electronic devices 38, 40, 42, 44 may each execute an operating system, examples of which may include but are not limited to, Android™, Apple® iOS®, Mac® OS X®; Red Hat® Linux®, Windows® Mobile, Chrome OS, Blackberry OS, Fire OS, or a custom operating system.

In some implementations, one or more of thermographic systems 22, 24, 26, 28 may be configured to effectuate some or all of the functionality of thermographic system 10 (and vice versa). Accordingly, in some implementations, thermographic system 10 may be a purely server-side application, a purely client-side application, or a hybrid server-side/client-side application that is cooperatively executed by one or more of thermographic systems 22, 24, 26, 28 and/or thermographic system 10.

In some implementations, one or more of thermographic systems 22, 24, 26, 28 may be configured to effectuate some or all of the functionality of imaging application 20 (and vice versa). Accordingly, in some implementations, imaging application 20 may be a purely server-side application, a purely client-side application, or a hybrid server-side/client-side application that is cooperatively executed by one or more of thermographic systems 22, 24, 26, 28 and/or imaging application 20. As one or more of thermographic systems 22, 24, 26, 28, thermographic system 10, and imaging application 20, taken singly or in any combination, may effectuate some or all of the same functionality, any description of effectuating such functionality via one or more of thermographic systems 22, 24, 26, 28, thermographic system 10, imaging application 20, or combination thereof, and any described interaction(s) between one or more of thermographic systems 22, 24, 26, 28, thermographic system 10, imaging application 20, or combination thereof to effectuate such functionality, should be taken as an example only and not to limit the scope of the disclosure.

In some implementations, one or more of users 46, 48, 50, 52 may access computer 12 and thermographic system 10 (e.g., using one or more of client electronic devices 38, 40, 42, 44) directly through network 14 or through secondary network 18. Further, computer 12 may be connected to network 14 through secondary network 18, as illustrated with phantom link line 54. Thermographic system 10 may include one or more user interfaces, such as browsers and textual or graphical user interfaces, through which users 46, 48, 50, 52 may access thermographic system 10. Further, in some implementations, thermographic systems 22, 24, 26, 28, may include standalone thermographic systems. In such implementations, users 46, 48, 50, 52 may access thermographic systems 22, 24, 26, 28 directly via respective client electronic devices 38, 40, 42, 44.

In some implementations, the various client electronic devices may be directly or indirectly coupled to network 14 (or network 18). For example, client electronic device 38 is shown directly coupled to network 14 via a hardwired network connection. Further, client electronic device 44 is shown directly coupled to network 18 via a hardwired network connection. Client electronic device 40 is shown wirelessly coupled to network 14 via wireless communication channel 56 established between client electronic device 40 and wireless access point (i.e., WAP) 58, which is shown directly coupled to network 14. WAP 58 may be, for example, an IEEE 802.11a, 802.11b, 802.11g, 802.11n, 802.11ac, Wi-Fi®, RFID, and/or Bluetooth™ (including Bluetooth™ Low Energy) device that is capable of establishing wireless communication channel 56 between client electronic device 40 and WAP 58. Client electronic device 42 is shown wirelessly coupled to network 14 via wireless communication channel 60 established between client electronic device 42 and cellular network/bridge 62, which is shown by example directly coupled to network 14.

In some implementations, some or all of the IEEE 802.11x specifications may use Ethernet protocol and carrier sense multiple access with collision avoidance (i.e., CSMA/CA) for path sharing. The various 802.11x specifications may use phase-shift keying (i.e., PSK) modulation or complementary code keying (i.e., CCK) modulation. Bluetooth™ (including Bluetooth™ Low Energy) is a telecommunications industry specification that allows, e.g., mobile phones, computers, smart phones, and other electronic devices to be interconnected using a short-range wireless connection. Other forms of interconnection (e.g., Near Field Communication (NFC)) may also be used.

In some implementations, various I/O requests (e.g., I/O request 15) may be sent from, e.g., thermographic systems 22, 24, 26, 28 to, e.g., computer 12. Examples of I/O request 15 may include, but are not limited to, data write requests (e.g., a request that content be written to computer 12) and data read requests (e.g., a request that content be read from computer 12).

In some implementations, as will be discussed below in greater detail, a thermographic system, such as thermographic system 10 of FIG. 1, may include but is not limited to a thermographic camera configured to capture a thermal image of a host body. The thermographic system may also include a computing device for receiving the thermal image of the host body, and detecting the presence of a cold-blooded animal on the host body based upon temperature information from the thermal image.

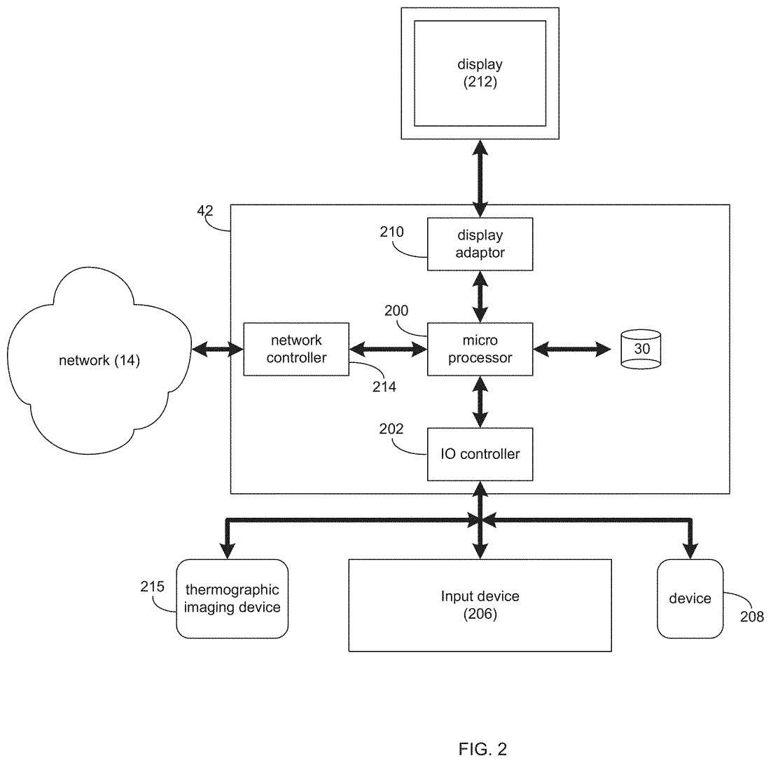

Referring also to the example implementation of FIG. 2, there is shown a diagrammatic view of client electronic device 42. While client electronic device 42 is shown in this figure, this is for example purposes only and is not intended to be a limitation of this disclosure, as other configurations are possible. Additionally, any computing device capable of executing, in whole or in part, a thermographic system may be substituted for client electronic device 42 (in whole or in part) within FIG. 2, examples of which may include but are not limited to computer 12 and/or one or more of client electronic devices 38, 40, 44, and/or an internal and/or external thermographic camera compatible with IOS/ANDROID cell phone operating systems, or other computing systems.

In some implementations, client electronic device 42 may include a processor (e.g., microprocessor 200) configured to, e.g., process data and execute the above-noted code/instruction sets and subroutines. Microprocessor 200 may be coupled via a storage adaptor to the above-noted storage device(s) (e.g., storage device 30). An I/O controller (e.g., I/O controller 202) may be configured to couple microprocessor 200 with various devices (e.g., via wired or wireless connection), such as input device 206 (including, but not limited to, a touch input device, such as a touch input display), pointing/selecting device (e.g., touchpad, touchscreen, mouse, etc.), custom device (e.g., device 215), USB ports, and printer ports. A display adaptor (e.g., display adaptor 210) may be configured to couple display 212 (e.g., touchscreen display, monitor(s), plasma, CRT, or LCD monitor(s), etc.) with microprocessor 200, while network controller/adaptor 214 (e.g., an Ethernet adaptor) may be configured to couple microprocessor 200 to the above-noted network 14 (e.g., the Internet or a local area network).

In some implementations, the custom device 215 may include a thermographic camera configured to capture a thermal image of a host body. An example of suitable thermographic imaging systems may include imaging systems available from FLIR Systems, Inc., such as the FLIR One thermal camera. It will be appreciated that other thermographic cameras and/or other thermal imaging systems may also suitably be used in connection with the present disclosure. An example thermographic system may capture the thermal image by utilizing, e.g., an internal and/or external thermographic camera that may be compatible with IOS/ANDROID cell phone operating systems, or other computing systems.

As will be discussed in greater detail below, in some implementations, a thermographic system consistent with the present disclosure may at least help with the detection of cold-blooded animal (such as ticks or the like), which may be disposed on a host (such as a person or an animal, like a dog). For example, in some embodiments a thermographic system may allow the heat signatures of mammals (e.g., a dog) to be identified in contrast to the heat signatures of cold-blooded animals (e.g., a tick) for purposes of detecting the presence and/or location of cold-blooded animals on a host body. In an example embodiment, a thermographic system may receive a thermal image of a host body. The thermal image may be used to at least help detect the presence of a cold-blooded animal on the host body based upon temperature information gathered from the thermal image. As early detection of the location of these cold-blooded animals may be critical for preventing disease and illness, in some embodiments a thermographic system may, e.g., provide a mechanism for efficiently detecting cold-blooded animals on a host body before the cold-blooded animal has time to infect the host body with, for example, a tick-borne pathogen, or provide preparation lead time to treat transmitted infections due to delayed detection. It will be appreciated that the computer processes described throughout may be implemented in an example practical application at least to improve technological processes of thermographic data gathering relative to detecting cold-blooded animals on a host body.

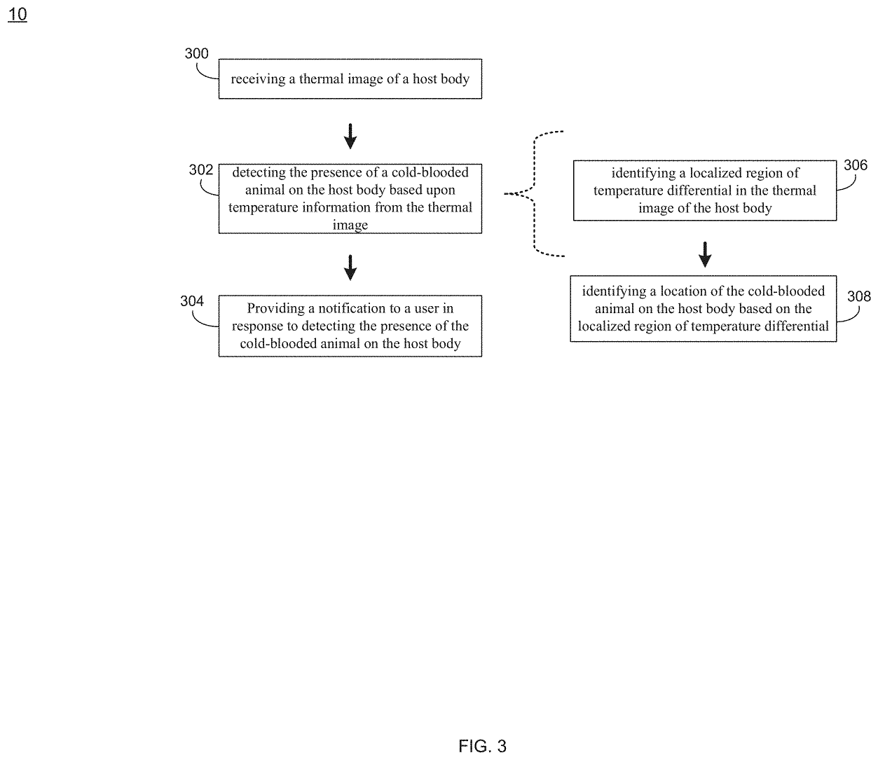

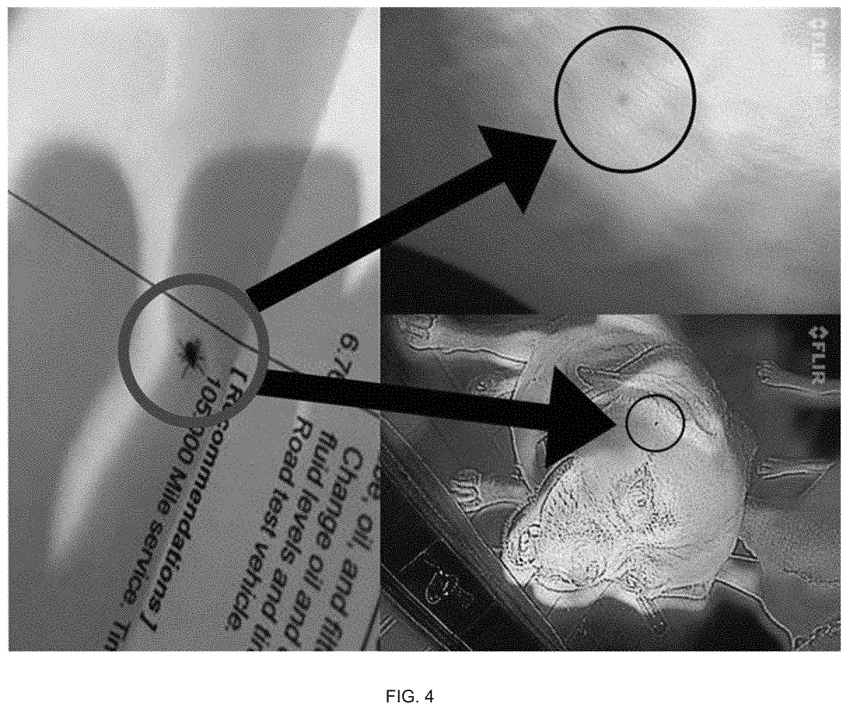

As discussed above, and referring also at least to the example implementations of FIGS. 3-4, in general, thermographic system 10 may receive 300 a thermal image of a host body. Thermographic system 10 may detect 302 the presence of a cold-blooded animal on the host body based upon temperature information from the thermal image. Consistent with the present disclosure, a “host” may be defined as a warm blooded animal including, but not limited to, mammals such as dogs, cats, and human beings, etc. A “cold-blooded animal” may be defined as a cold blooded animal including, but not limited to, ticks, mites, bed bugs, or other insects of the like. While the present disclosure may generally discuss identifying a tick as the cold blooded animal, it will be appreciated that the principles herein may be applied to identifying other cold-blooded animals. As will be elaborated upon in greater detail, thermal imaging techniques may be used to identify the heat signatures of mammals in contrast to the heat signatures of cold-blooded animals.

As mentioned above, thermographic system 10 may receive 300 a thermal image of a host body. The thermal image may be received directly from a thermographic camera system and/or may be received from a storage device, e.g., that may be associated with a thermographic camera, that may be associated with a computing device (e.g., which may include a computing device detecting 302 the presence of the cold blooded animal), and/or from a standalone storage device and/or a storage device associated with another computing device. In various embodiments, the thermal image received 300 by the thermographic system 10 may have been taken with any suitable thermographic camera and/or thermographic imaging element. As generally discussed above, the thermographic camera used to take the thermal image of the host body may include any suitable thermographic camera and/or thermographic imaging element. Examples of suitable thermographic cameras and/or thermographic imaging elements may include standalone thermographic cameras and/or thermographic imaging modules attachable to a smartphone (e.g., FLIR E5 and FLIR ONE, respectively, both available from FLIR Systems). It will be appreciated that various additional and/or alternative thermographic cameras and/or thermographic imaging modules may be utilized, having a variety of different configurations and/or available from a variety of difference manufacturers.

In some implementations, thermographic system 10 may detect 302 the presence of a cold-blooded animal on the host body based upon temperature information from the thermal image. For example, and as will be elaborated upon in greater detail below, the thermal temperature of the host body and/or the cold-blooded animal may be determined using a thermal imaging camera, e.g., which may generally provide a thermal image of the host body and/or the cold-blooded animal.

In some implementations, detecting 302 the presence of the cold-blooded animal on the host body may include identifying 306 a localized region of temperature differential in the thermal image of the host body. Consistent with the previous example, the detected temperatures in the thermal image associated with various portions of the host body and/or cold-blooded animal may be depicted using different colors or color gradiations. Thermal images of a host body may include different colors or color gradiations representative of various temperatures as they relate to the various regions of a host body and a cold-blooded animal. For example, detecting 302 the presence of the cold-blooded animal on the host body includes identifying 308 a location of the cold-blooded animal on the host body based on the localized region of temperature differential. In some embodiments, the magnitude of the temperature differential between the host temperature and the localized region exhibiting a lower temperature may be selected based upon, for example, the host temperature, the ambient temperature at the time the image is taken, the ambient temperature of the location where the host may have come into contact with the cold-blooded animal, accuracy of the thermal imaging system, resolution of thermal imaging system (e.g., one or more of temperature detection resolution and/or image quality resolution), etc. It will be appreciated that various additional and/or alternative criteria may be considered in selecting a temperature difference considered as being indicative of a cold-blooded animal.

In some implementations, identifying 308 the location of the cold-blooded animal on the host body includes utilizing a temperature delta between the host body and the cold-blooded animal. For example, when a cold-blooded animal is situated on a host, body, thermal imaging may identify and locate the cold-blooded animal utilizing the temperature difference between host and the cold-blooded animal. As will be appreciated, the temperature delta may be identifiable from the thermal image of the host as a location having a different color, e.g., representing a difference in temperature.

Continuing with the foregoing, detecting 302 the presence of the cold-blooded animal on the host body includes detecting the localized region of temperature differential having a size corresponding to a size of the cold-blooded animal of interest. For example, and with particular reference to FIG. 4, an illustrative example embodiment of a thermal image consistent with the present disclosure is shown, particularly depicting a thermal image of a host body (i.e., dog) taken 15 minutes after the host body was exposed to the habitat of the cold-blooded animal. Utilizing, e.g., a FlirOne Thermographic Camera, the thermal image depicts the thermographic heat signatures of two dogs, including a small short haired puggle (e.g., “host body”) having a body temperature of 102° F., and a medium size beagle mix having a thermographic heat signature of 102° F. The thermal image of the host body may be analyzed to identify localized regions exhibiting a lower temperature than the host, and having a size generally corresponding to the size of a cold-blooded animal of interest. The illustrated example embodiment is shown including the cold-blooded animal of interest; which may be, e.g., a tick, a mite, a bed bug, or other insect of the like, of considerably smaller size in comparison to a host body to which the cold-blooded animal is attached. It will be appreciated that the size generally corresponding to the size of a cold blooded animal of interest may include a minimum size, a maximum size, and/or a size range that may encompass cold-blooded animals of interest. Further, in some embodiments, the size may be selected based upon, at least in part, characteristics of the thermal imaging system, such as camera image quality resolution, camera temperature resolution, or the like. Various additional and/or alternative factors may be utilized in determining a relevant size range.

In some implementations, as generally discussed, identifying 308 the location of the cold-blooded animal on the host body includes detecting one or more localized regions exhibiting a lower temperature than the host body. For example, the thermal image may produce localized regions having a cooler color gradient emitted by the cold-blooded animal, against a warmer color gradient emitted by the host body. For example, the one or more localized regions exhibiting a lower temperature than the host body is selected based upon, at least in part, the thermal temperature of the host body. For example, the normal body temperature for a warm blooded animal, e.g., a dog, may fall within the range of 100° F. to 102.5° F. (about 38° C. to 39.2° C.). A human may have a heat signature of approximately 98.5° F. A cat's normal body temperature may range from 100.5 to 102.5 degrees. In comparison, the body temperature for a cold blooded animal, e.g., nymph tick, may have a thermographic heat signature of 89° F. Accordingly, the cold-blooded animal may emit a considerably cooler color gradient than the color gradient emitted from the host body.

In some implementations, thermographic system 10 may provide 304 a notification to a user in response to detecting 302 the presence of the cold-blooded animal on the host body. For example, the notification to a user in response to detecting 302 the presence of the cold-blooded animal on the host body may include providing an alarm and/or notification to a user once the cold-blooded animal has been located. Further, in some embodiments, providing 304 a notification to a user may include identifying a location of the cold-blooded animal on the host body. For example, a location of the cold-blooded animal on the host body may be highlighted, identified with an indicator (such as an arrow pointing to the cold-blooded animal, a circle or box around the cold-blooded animal, etc.), within the thermal image of the host body. For example, during and/or after detecting 302 the presence of the cold-blooded animal, the thermal image of the host body may be displayed on a display device, such as the display of a smartphone or other computing device. Upon detecting 302 the cold-blooded animal on the host body, the thermographic system 10 may provide 304 a notification of the presence of the cold-blooded animal. In some embodiments, providing the notification may include providing an indicator of the location of the cold-blooded animal in the displayed thermal image.

The terminology used herein is for the purpose of describing particular implementations only and is not intended to be limiting of the disclosure. As used herein, the singular forms “a”, “an” and “the” are intended to include the plural forms as well, unless the context clearly indicates otherwise. As used herein, the language “at least one of A, B, and C” (and the like) should be interpreted as covering only A, only B, only C, or any combination of the three, unless the context clearly indicates otherwise. It will be further understood that the terms “comprises” and/or “comprising,” when used in this specification, specify the presence of stated features, integers, steps (not necessarily in a particular order), operations, elements, and/or components, but do not preclude the presence or addition of one or more other features, integers, steps (not necessarily in a particular order), operations, elements, components, and/or groups thereof.

The corresponding structures, materials, acts, and equivalents (e.g., of all means or step plus function elements) that may be in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications, variations, substitutions, and any combinations thereof will be apparent to those of ordinary skill in the art without departing from the scope and spirit of the disclosure. The implementation(s) were chosen and described in order to explain the principles of the disclosure and the practical application, and to enable others of ordinary skill in the art to understand the disclosure for various implementation(s) with various modifications and/or any combinations of implementation(s) as are suited to the particular use contemplated.

A number of implementations have been described. Having thus described the disclosure of the present application in detail and by reference to implementation(s) thereof, it will be apparent that modifications, variations, and any combinations of implementation(s) (including any modifications, variations, substitutions, and combinations thereof) are possible without departing from the scope of the disclosure defined in the appended claims.

What is claimed is:

1. A computer-implemented method comprising:
receiving a thermal image of a host body; and
detecting the presence of a cold-blooded animal on the host body based upon temperature information from the thermal image.

2. The computer-implemented method according to claim 1 wherein detecting the presence of the cold-blooded animal on the host body includes identifying a localized region of temperature differential in the thermal image of the host body.

3. The computer-implemented method according to claim 2 wherein detecting the presence of the cold-blooded animal on the host body includes identifying a location of the cold-blooded animal on the host body based on the localized region of temperature differential.

4. The computer-implemented method according to claim 3 wherein identifying the location of the cold-blooded animal on the host body includes utilizing a temperature delta between the host body and the cold-blooded animal.

5. The computer-implemented method according to claim 1, further comprising:
providing a notification to a user in response to detecting the presence of the cold-blooded animal on the host body.

6. The computer-implemented method according to claim 2 wherein detecting the presence of the cold-blooded animal on the host body includes detecting the localized region of temperature differential having a size corresponding to a size of the cold-blooded animal of interest.

7. The computer-implemented method according to claim 3 wherein identifying the location of the cold-blooded animal on the host body includes detecting one or more localized regions exhibiting a lower temperature than the host body.

8. The computer-implemented method according to claim 7 wherein the one or more localized regions exhibiting a lower temperature than the host body is selected based upon, at least in part, the thermal temperature of the host body.

9. A thermographic system comprising:
a thermographic camera configured to capture a thermal image of a host body; and
a computing device for receiving the thermal image of the host body, and detecting the presence of a cold-blooded animal on the host body based upon temperature information from the thermal image.

10. The thermographic system according to claim 9 wherein detecting the presence of the cold-blooded animal on the host body includes identifying a localized region of temperature differential in the thermal image of the host body.

11. The thermographic system according to claim 10 wherein detecting the presence of the cold-blooded animal on the host body includes identifying a location of the cold-blooded animal on the host body based on the localized region of temperature differential.

12. The thermographic system according to claim 11 wherein identifying the location of the cold-blooded animal on the host body includes utilizing a temperature delta between the host body and the cold-blooded animal.

13. The thermographic system according to claim 9, further comprising:
providing a notification to a user in response to detecting the presence of the cold-blooded animal on the host body.

14. The thermographic system according to claim 10 wherein detecting the presence of the cold-blooded animal on the host body includes detecting the localized region of temperature differential having a size corresponding to a size of the cold-blooded animal of interest.

15. The thermographic system according to claim 11 wherein identifying the location of the cold-blooded animal on the host body includes detecting one or more localized regions exhibiting a lower temperature than the host body.

16. The thermographic system according to claim 15 wherein the one or more localized regions exhibiting a lower temperature than the host body is selected based upon, at least in part, the thermal temperature of the host body.

17. A computer program product residing on a computer readable storage medium having a plurality of instructions stored thereon which, when executed across one or more processors, causes at least a portion of the one or more processors to perform operations comprising:
receiving a thermal image of a host body; and
detecting the presence of a cold-blooded animal on the host body based upon temperature information from the thermal image.

18. The computer program product according to claim 17 wherein detecting the presence of the cold-blooded animal on the host body includes identifying a localized region of temperature differential in the thermal image of the host body.

19. The computer program product according to claim 18 wherein detecting the presence of the cold-blooded animal on the host body includes identifying a location of the cold-blooded animal on the host body based on the localized region of temperature differential.

20. The computer program product according to claim 19 wherein identifying the location of the cold-blooded animal on the host body includes detecting one or more localized regions exhibiting a lower temperature than the host body.
